(19) United States
(12) Patent Application Publication
(10) Pub. No.: US 2025/0295515 A1
(43) Pub. Date: Sep. 25, 2025

(54) DEVICE FOR ASSISTING MUSCULAR STRENGTH

(71) Applicants: Hyundai Motor Company, Seoul (KR); Kia Corporation, Seoul (KR)

(72) Inventors: Seok Ryung Kwon, Anyang-si (KR); Beom Su Kim, Yongin-si (KR); Hyun Seop Lim, Anyang-si (KR); Ju Young Yoon, Suwon-si (KR)

(21) Appl. No.: 18/773,871

(22) Filed: Jul. 16, 2024

(30) Foreign Application Priority Data
Mar. 20, 2024 (KR) 10-2024-0038618

(51) Int. Cl. A61F 5/02

(52) U.S. Cl. CPC A61F 5/02

(57) ABSTRACT

A device for assisting muscular strength can include a support part, link parts including first and second link parts each having one side fixed to the support part, and wire parts configured to connect the two link parts adjacent to each other among the plurality of link parts, in which the wire parts include a first wire part having one side connected to a base region of the first link part and the other side connected to a base region of the second link part, and a second wire part having one side connected to the base region of the second link part and the other side connected to a protruding region of the first link part.

### CROSS-REFERENCE TO RELATED APPLICATIONS

This application claims priority to and the benefit of Korean Patent Application No. 10-2024-0038618 filed in the Korean Intellectual Property Office on Mar. 20, 2024, the entire contents of which are incorporated herein by reference.

### TECHNICAL FIELD

The present disclosure relates to a device for assisting muscular strength.

### BACKGROUND

Devices for assisting muscular strength, which generate assistive power for assisting muscular strength, such as a person's waist muscles and the like, may be broadly classified into a so-called hard type device configured to generate assistive power for assisting muscular strength by actively creating a force from a power source, and a so-called soft type device configured to passively generate assistive power in accordance with a change in posture of a wearer. Among the devices, the soft type device for assisting muscular strength immediately generates assistive power when the wearer's posture changes. Therefore, the soft type device for assisting muscular strength has an advantage in that a separate power source for generating assistive power is not required, the assistive power having a consistent size and direction is generated in accordance with the change in posture of the wearer, and heterogeneity caused by the assistive power is relatively low. In contrast, the soft type device for assisting muscular strength has a problem in that a magnitude of the assistive power is approximately lower than that of the hard type device for assisting muscular strength. In addition, the soft type device for assisting muscular strength in the related art has a problem in that a direction in which the assistive power for assisting muscular strength is applied is limited, and the assistive power cannot be sufficiently generated in various directions in response to various changes in postures of the wearer. For example, the waist device for assisting muscular strength in the related art, which assists muscular strength of the wearer's waist, generates assistive power in a direction in which the wearer's waist is stretched when the wearer bends the waist. However, when the wearer twists the waist, the waist device for assisting muscular strength cannot generate assistive power in a direction opposite to a direction in which the waist is twisted.

Therefore, there is a need to develop a device for assisting muscular strength that may have the advantages of the soft type device for assisting muscular strength in the related art in an intact manner, generate higher assistive power, and generate assistive power in various directions in response to various changes in postures of a wearer.

### SUMMARY

The present disclosure relates to a device for assisting muscular strength, and more particularly, to a device for assisting muscular strength that may be used to assist a person's waist muscles.

An embodiment of the present disclosure can provide a device for assisting muscular strength with a novel structure, which is capable of generating higher assistive power in comparison with a device in the related art and generating assistive power in various directions in response to various changes in postures of a wearer.

To achieve the above-mentioned capabilities, an embodiment of the present disclosure can provide a device for assisting muscular strength, the device can include: a support part including a section extending along a straight or curved line; link parts including a first link part having one side fixed to the support part, and a second link part provided below the first link part; and wire parts configured to connect the first link part and the second link part, in which the first and second link parts each includes a base region and a protruding region protruding downward from the base region, and in which the wire parts include a first wire part having one side connected to the base region of the first link part and the other side connected to the base region of the second link part, and a second wire part having one side connected to the base region of the second link part and the other side connected to the protruding region of the first link part.

When a direction in which the first link part and the second link part face the support part is defined as a forward direction, the first wire part may include: a first-first wire configured to connect a front region of the base region of the first link part and a front region of the base region of the second link part; and a first-second wire provided to face the first-first wire with the protruding region of the first link part interposed therebetween.

When a direction in which the first link part and the second link part face the support part is defined as a forward direction, the second wire part may include: a second-first wire configured to connect the protruding region of the first link part and a portion of the base region of the second link part that is positioned forward of the protruding region of the first link part; and a second-second wire configured to connect the protruding region of the first link part and a portion of the base region of the second link part that is positioned rearward of the protruding region of the first link part.

The second wire parts may be respectively provided on left and right regions of the first wire part.

The second wire part provided on the right region of the first wire part and the second wire part provided on the left region of the first wire part may be structured to be symmetric with respect to the first wire part.

Through-holes may be respectively formed, in an upward/downward direction, through the base region of the first link part and the base region of the second link part, any one of a lower end of the protruding region of the first link part and an upper end of the protruding region of the second link part may include a curvedly protruding portion protruding while having a curved shape, and the other of the lower end of the protruding region of the first link part and the upper end of the protruding region of the second link part may include a curvedly recessed portion having a shape corresponding to the curvedly protruding portion.

The curvedly protruding portion may be a ball joint member.

The curvedly protruding portion may be provided at the lower end of the protruding region of the first link part, and the curvedly recessed portion may be provided at the upper end of the protruding region of the second link part.

The protruding region may include a first extension section extending in a direction intersecting the upward/downward direction, and the curvedly recessed portion may be provided on an upper surface of the first extension section of the protruding region of the second link part.

The protruding region may further include: a connection section extending downward from one side of the first extension section; and a second extension section connected to a lower end of the connection section and extending in the direction intersecting the upward/downward direction, and the curvedly protruding portion may be provided on a lower surface of the second extension section of the protruding region of the first link part.

A longitudinal direction of the first extension section and a longitudinal direction of the second extension section may be parallel to each other.

The connection section may include: a first connection section inclinedly extending downward from one longitudinal end of the first extension section; and a second connection section inclinedly extending downward from the other longitudinal end of the first extension section, and the second extension section may be connected to a lower end of the first connection section and a lower end of the second connection section.

Protruding connection portions, to which the second wire part is connected, may be provided at one longitudinal side of the second extension section, and one side of the second-first wire and one side of the second-second wire may be respectively connected to the protruding connection portions.

The protruding connection portions may be respectively provided at two opposite longitudinal sides of the second extension section, and the lower end of the connection section may be provided between the two protruding connection portions respectively provided at the two opposite longitudinal sides of the second extension section.

The support part may be provided to be tightly attached to or in contact with a wearer's waist.

According to an embodiment of the present disclosure, it is possible to provide the device for assisting muscular strength with a novel structure, which is capable of generating higher assistive power in comparison with a device in the related art and generating assistive power in various directions in response to various changes in postures of a wearer.

### BRIEF DESCRIPTION OF THE DRAWINGS

FIG. 1 is a perspective view of an overall structure of a device for assisting muscular strength according to an embodiment of the present disclosure.

FIG. 2 is an enlarged view illustrating a shape of two adjacent link parts of the device for assisting muscular strength according to an embodiment of the present disclosure when viewed from a first side.

FIG. 3 is an enlarged view illustrating a shape of the two adjacent link parts of the device for assisting muscular strength according to an embodiment of the present disclosure when viewed from a second side.

FIG. 4 is a cross-sectional view illustrating a vertical cross-sectional shape of the link part of the device for assisting muscular strength according to an embodiment of the present disclosure.

### DETAILED DESCRIPTION OF ILLUSTRATIVE EMBODIMENTS

Hereinafter, a device for assisting muscular strength according to an example embodiment of the present disclosure will be described with reference to the drawings.

### Device for Assisting Muscular Strength

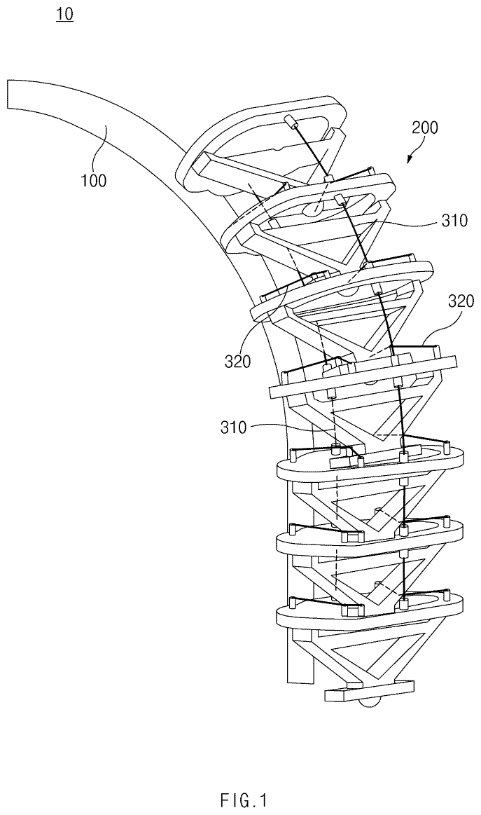

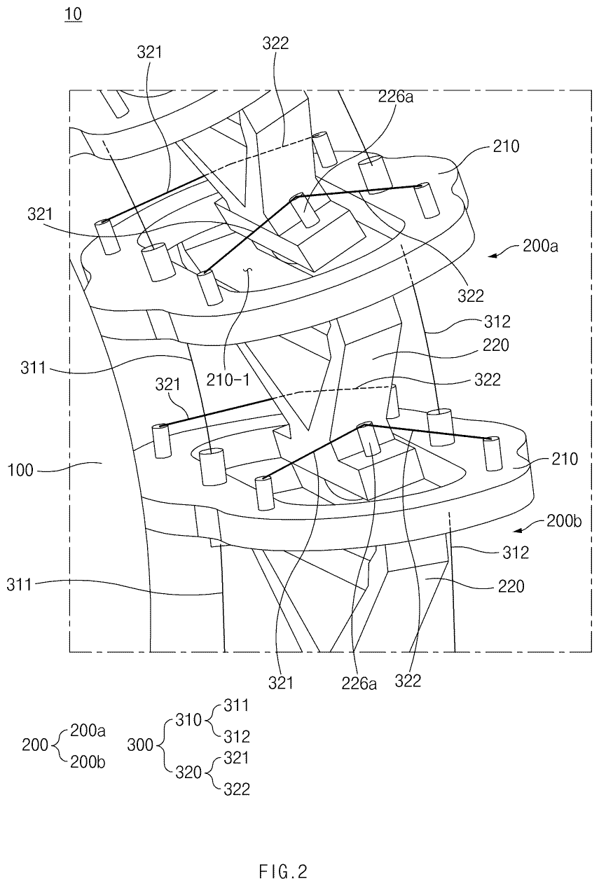

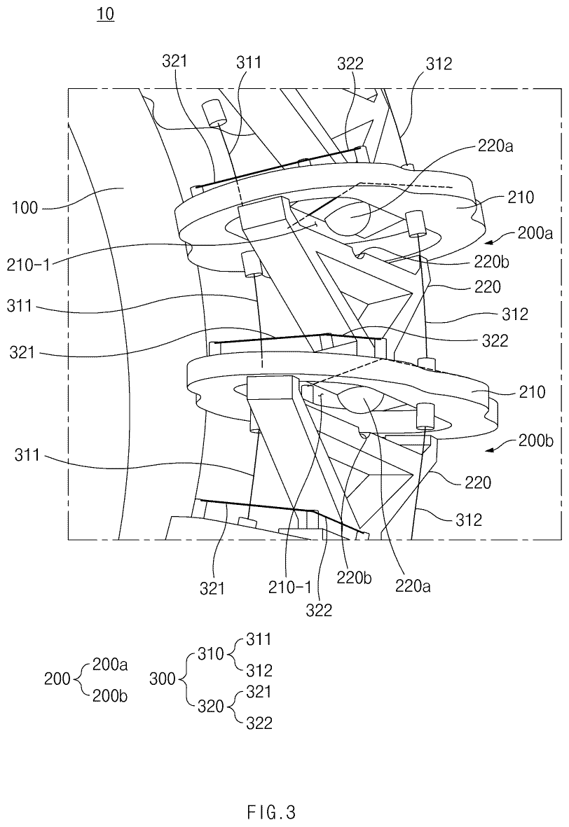

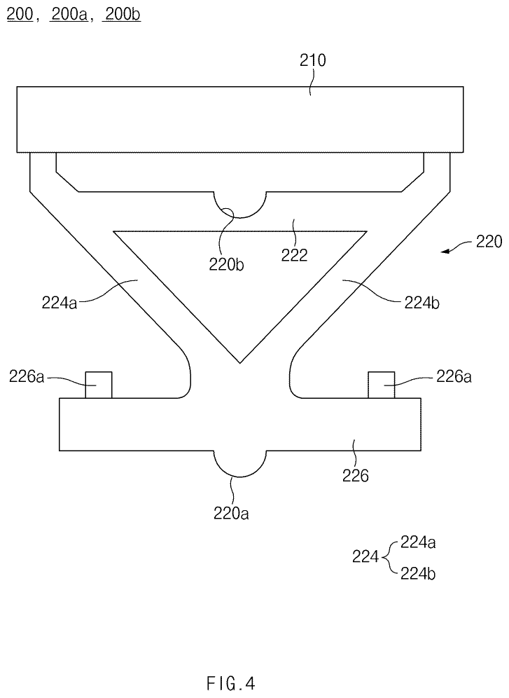

FIG. 1 is a perspective view of an overall structure of a device for assisting muscular strength according to an example embodiment of the present disclosure. FIG. 2 is an enlarged view illustrating a shape of two adjacent link parts of the device for assisting muscular strength according to an example embodiment of the present disclosure when viewed from a first side. FIG. 3 is an enlarged view illustrating a shape of the two adjacent link parts of the device for assisting muscular strength according to an example embodiment of the present disclosure when viewed from a second side. FIG. 4 is a cross-sectional view illustrating a vertical cross-sectional shape of the link part of the device for assisting muscular strength according to an example embodiment of the present disclosure.

The device for assisting muscular strength according to an embodiment of the present disclosure may be a device for assisting strength of a wearer's muscles. The device for assisting muscular strength according to the example embodiment of the present disclosure may be a device for assisting strength of a wearer's waist muscles. In this case, the device for assisting muscular strength according to an embodiment of the present disclosure may be a device having a structure capable of reducing a load to be applied to the wearer's waist by providing assistive power in a direction in which the waist is stretched when the wearer bends the waist forward. In addition, as described below, in a posture in which the wearer stands while stretching the waist, the device for assisting muscular strength according to an embodiment of the present disclosure may additionally provide assistive power for assisting a load applied to the waist, such that the device for assisting muscular strength may provide assistive power to the waist even in a posture in which the wearer moves or stands while holding a heavy object.

With reference to FIGS. 1 to 4, a device 10 for assisting muscular strength according to an embodiment of the present disclosure may include a support part 100 including a section extending along a straight or curved line. In case that the device 10 for assisting muscular strength according to the present disclosure is the device for assisting the strength of the wearer's waist muscles as described above, the support part 100 may be provided to be tightly attached or in contact with the wearer's waist. Therefore, the support part 100 may have a shape corresponding to the wearer's waist.

The support part 100 may be configured to be deformed in shape corresponding to a shape of the wearer's waist. That is, the support part 100 may be made of a material that may be deformed to correspond to a state of the waist of the wearer who wears the device 10 for assisting muscular strength. Alternatively, the support part 100 may have a structure that may be deformed to correspond to the state of the waist of the wearer. For example, the support part 100 may be made of a material that may be elastically deformed when the support part 100 is bent and twisted by an external force. Alternatively, the support part 100 may be made of a woven fabric material such as a fabric that may be tightly attached to the wearer's waist and freely deformed in shape depending on the state of the waist. In the present specification, when the wearer wears the device 10 for assisting muscular strength, a front side, a rear side, a lateral side, an upper side, and a lower side of the wearer are respectively defined as a front side, a rear side, a lateral side, an upper side, and a lower side of the device 10 for assisting muscular strength.

The device 10 for assisting muscular strength according to an embodiment of the present disclosure may include a plurality of link parts 200 each having one side fixed to the support part 100. For example, FIG. 1 illustrates a state in which the device 10 for assisting muscular strength includes seven link parts 200. More specifically, a front region of each of the plurality of link parts 200 may be fixedly coupled to the support part 100, and the plurality of link parts 200 may be configured to be spaced apart from one another in an upward/downward direction. Hereinafter, in the present specification, the description of the link parts 200 of the device 10 for assisting muscular strength according to an embodiment of the present disclosure will be focused on a first link part 200a, which is one of the plurality of link parts 200 and has one side fixed to the support part 100, and a second link part 200b provided to be closest to the first link part 200a among the plurality of link parts provided below the first link part 200a. However, the following description of the first link part 200a, the second link part 200b, and a relationship between the first link part 200a and the second link part 200b may be equally applied to the two link parts adjacent to each other in the upward/downward direction and included in the device 10 for assisting muscular strength according to the present disclosure.

The plurality of link parts 200 provided in the device 10 for assisting muscular strength may each include a plurality of regions. More specifically, the plurality of link parts 200, which includes the first link part 200a and the second link part 200b, may each include a base region 210 and a protruding region 220 protruding downward from the base region 210. The base region 210 may have a plate shape having an approximately constant thickness in the upward/downward direction. However, the shape of the base region 210 is not limited to the plate shape. Example detailed shapes of the base region 210 and the protruding region 220 will be described below.

With continued reference to FIGS. 1 to 3, the device 10 for assisting muscular strength according to an embodiment of the present disclosure may further include wire parts 300 configured to connect the first link part 200a and the second link part 200b. The wire part 300 may be configured to generate assistive power for assisting the strength of the wearer's waist muscles by use of tension of wires. More specifically, the wire part 300 may include a plurality of wires. A length of the wire may vary depending on a change in relative position between the two adjacent link parts 200. Therefore, the assistive power for assisting the strength of the wearer's waist muscles may be generated by a tensile force of the wire generated by the change in relative position between the two adjacent link parts 200. The plurality of wires included in the wire part 300 may include or be made of a material that may be elastically deformed by an external force.

The plurality of wires of the wire part 300 may be distinguished depending on which regions of the two adjacent link parts 200 are connected to one another. More specifically, the wire part 300 may include a first wire part 310 having one side connected to the base region 210 of the first link part 200a and the other side connected to the base region 210 of the second link part 200b, and a second wire part 320 having one side connected to the base region 210 of the second link part 200b and the other side connected to the protruding region 220 of the first link part 200a. The first wire part 310 and the second wire part 320 may be configured separately from each other.

According to an embodiment of the present disclosure, the first wire part 310 and the second wire part 320 may each include the plurality of wires. More specifically, assuming that a direction in which the first link part 200a and the second link part 200b face the support part 100 is a forward direction, the first wire part 310 may include a first-first wire 311 configured to connect a front region of the base region 210 of the first link part 200a and a front region of the base region 210 of the second link part 200b, and a first-second wire 312 provided to face the first-first wire 311 with the protruding region 220 of the first link part 200a interposed therebetween.

The first-first wire 311 and the first-second wire 312 may each be configured to generate the assistive power for assisting the strength of the wearer's waist muscles by generating a tensile force when the wearer of the device 10 for assisting muscular strength bends the waist forward. That is, with reference to FIGS. 1 to 3, when the wearer of the device 10 for assisting muscular strength bends the waist forward, the two link parts 200, e.g., the first link part 200a and the second link part 200b, which are spaced apart from each other in the upward/downward direction and adjacent to each other, are moved away from each other. Therefore, the first-first wire 311 and the first-second wire 312, which connect the base region 210 of the first link part 200a and the base region 210 of the second link part 200b, are stretched. Therefore, tensile forces are applied to the first-first wire 311 and the first-second wire 312, and the assistive power is generated in a direction (i.e., a direction in which the wearer's waist is stretched) opposite to the direction in which the two adjacent link parts 200, e.g., the first link part 200a and the second link part 200b are moved away from each other. In the present specification, the front region of the base region 210 of the link part 200 may be understood as a region positioned forward of the protruding region of another link part adjacent to the link part 200, and the rear region of the base region 210 of the link part 200 may be understood as a region positioned rearward of the protruding region of another link part adjacent to the link part 200.

As illustrated in FIGS. 1 to 3, in a state in which the wearer stands while wearing the device 10 for assisting muscular strength, an angle of each of the first-first wire 311 and the first-second wire 312 with respect to the upward/downward direction may be smaller than an angle of each of the first-first wire 311 and the first-second wire 312 with respect to a horizontal direction, and an angle of each of a second-first wire 321 and a second-second wire 322 with respect to the upward/downward direction may be larger than an angle of each of the second-first wire 321 and the second-second wire 322 with respect to the horizontal direction. This configuration may be understood as a configuration in which the first-first wire 311 and the first-second wire 312 are approximately parallel to the upward/downward direction or extend to be close to the upward/downward direction, whereas the second-first wire 321 and the second-second wire 322 are approximately parallel to the horizontal direction or extend to be close to the horizontal direction. Therefore, according to an embodiment of the present disclosure, the first wire part 310 including the first-first wire 311 and the first-second wire 312 may generate a tensile force in parallel with the upward/downward direction or in a direction close to the upward/downward direction, whereas the second wire part 320 including the second-first wire 321 and the second-second wire 322 may generate a tensile force in parallel with the horizontal direction or in a direction close to the horizontal direction.

The second wire part 320 may include the second-first wire 321 configured to connect the protruding region 220 of the first link part 200a and a portion of the base region 210 of the second link part 200b, which is positioned forward of the protruding region 220 of the first link part 200a, and the second-second wire 322 configured to connect the protruding region 220 of the first link part 200a and a portion of the base region 210 of the second link part 200b, which is positioned rearward of the protruding region 220 of the first link part 200a. More specifically, the second wire parts 320 may be respectively provided on left and right regions of the first wire part 310.

Like the first-first wire 311 and the first-second wire 312, the second-first wire 321 may be configured to generate a tensile force when the wearer of the device 10 for assisting muscular strength bends the waist forward. That is, with reference to FIGS. 1 to 3, when the wearer of the device 10 for assisting muscular strength bends the waist forward, a distance in a forward/rearward direction between the front region of the base region 210 of the second link part 200b and the protruding region 220 of the first link part 200a increases, such that the second-first wire 321 is stretched. Therefore, a tensile force is applied to the second-first wire 321, and the assistive power is generated in the direction opposite to the direction in which the two adjacent link parts 200, e.g., the first link part 200a and the second link part 200b are moved away from each other.

The second-second wire 322 may be configured to generate a tensile force when the wearer of the device 10 for assisting muscular strength bends the waist rearward. That is, with reference to FIGS. 1 to 3, when the wearer of the device 10 for assisting muscular strength performs an extension motion while bending the waist rearward, a distance in the forward/rearward direction between the rear region of the base region 210 of the second link part 200b and the protruding region of the first link part 200a increases, such that the second-second wire 322 is stretched. Therefore, a tensile force is applied to the second-second wire 322, and the assistive power is generated in the direction opposite to the direction in which the two adjacent link parts 200, e.g., the first link part 200a and the second link part 200b are moved away from each other. In addition, the second-second wire 322 may serve to stably maintain a relative position between the two adjacent link parts 200 of the device 10 for assisting muscular strength even in a situation in which the above-mentioned assistive power is not required.

According to the above-mentioned structure of the present disclosure, in case that the wearer wears the device 10 for assisting muscular strength according to an embodiment of the present disclosure on the waist, the assistive power may be generated in a direction opposite to a direction in which the waist is twisted not only when the wearer bends the waist but also when the wearer twists the waist.

According to an embodiment of the present disclosure, when the wearer wears the device 10 for assisting muscular strength and twists the waist, the support part 100 tightly attached to the wearer's waist is also twisted, and a relative rotational motion occurs between the plurality of link parts 200 positioned adjacent to one another and provided in the vicinity of a region in which the waist is twisted. In this case, a relative rotational motion occurs between the link part, which is positioned at an upper side between the two adjacent link parts 200, e.g., the protruding region 220 of the first link part 200a and the link part, which is positioned at a lower side between the two adjacent link parts 200, e.g., the base region 210 of the second link part 200b. Therefore, a length of any one of the second-first wire 321 and the second-second wire 322, which connect the two adjacent link parts 200, increases, such that the tensile force is generated. Because the tensile force is applied in the direction opposite to the direction in which the wearer's waist is twisted, the tensile force may be applied as assistive power required to restore the twisted waist of the wearer to an original state.

Therefore, the device 10 for assisting muscular strength according to an embodiment of the present disclosure may generate assistive power in various directions in response to various changes in postures of the wearer. In particular, in case that the wearer wears the device 10 for assisting muscular strength and twists the waist while simultaneously bending the waist, the device 10 for assisting muscular strength may generate the assistive power for stretching the waist and simultaneously generate the assistive power required to restore the twisted waist to the original state.

As described above, the second wire parts 320 may be respectively provided on the left and right regions of the first wire part 310. In this case, the second wire part 320 provided on the right region of the first wire part 310 and the second wire part 320 provided on the left region of the first wire part 310 may be structured to be symmetric with respect to the first wire part 310. More specifically, the second-first wire 321 of the second wire part 320 provided on the right region of the first wire part 310 and the second-first wire 321 of the second wire part 320 provided on the left region of the first wire part 310 may be configured to be symmetric with respect to the first wire part 310. The second-second wire 322 of the second wire part 320 provided on the right region of the first wire part 310 and the second-second wire 322 of the second wire part 320 provided on the left region of the first wire part 310 may be configured to be symmetric with respect to the first wire part 310.

With continued reference to FIGS. 1 to 3, among the plurality of link parts 200 provided in the device 10 for assisting muscular strength according to an embodiment of the present disclosure, the protruding regions 220 of the two adjacent link parts 200 may be provided to directly face each other.

To achieve the above-mentioned structure, according to an embodiment of the present disclosure, a through-hole 210-1 may be formed in the base region 210 of the link part 200. That is, according to an embodiment of the present disclosure, the through-holes 210-1 may be respectively formed, in the upward/downward direction, through the base region 210 of the first link part 200a and the base region 210 of the second link part 200b. The protruding region 220 of the first link part 200a and the protruding region 220 of the second link part 200b may directly face each other through the through-hole 210-1 of the second link part 200b.

More specifically, a lower end of the protruding region 220 of the first link part 200a and an upper end of the protruding region 220 of the second link part 200b may come into contact with each other or be spaced apart from each other depending on a degree to which the support part 100 is curved. More specifically, when the support part 100 is curved forward to a predetermined or set degree or higher, the lower end of the protruding region 220 of the first link part 200a and the upper end of the protruding region 220 of the second link part 200b may be spaced apart from each other. When the support part 100 is curved forward to a degree lower than the predetermined or set degree, the lower end of the protruding region 220 of the first link part 200a and the upper end of the protruding region 220 of the second link part 200b may be in contact with each other.

In case that the lower end of the protruding region 220 of the first link part 200a and the upper end of the protruding region 220 of the second link part 200b are in contact with each other, the first link part 200a may be supported by the second link part 200b, and the wearer of the device 10 for assisting muscular strength may be in a state in which the waist is stretched. Therefore, the device 10 for assisting muscular strength according to an embodiment of the present disclosure may not only provide the assistive power for assisting the strength of the waist muscles when the wearer's waist is bent, but also provide the supporting force for supporting the waist in the state in which the wearer's waist is stretched. Therefore, it is possible to effectively support the wearer's waist in a state in which the wearer stands while holding an object or the wearer moves while holding an object.

To provide the above-mentioned supporting force, according to an embodiment of the present disclosure, any one of the lower end of the protruding region 220 of the first link part 200a and the upper end of the protruding region 220 of the second link part 200b may include a curvedly protruding portion 220a protruding while having a curved shape, and the other of the lower end of the protruding region 220 of the first link part 200a and the upper end of the protruding region 220 of the second link part 200b may include a curvedly recessed portion 220b having a shape corresponding to the curvedly protruding portion 220a. For example, the curvedly protruding portion 220a may be a ball joint member. In case that the lower end of the protruding region 220 of the first link part 200a and the upper end of the protruding region 220 of the second link part 200b are in contact with each other, the curvedly protruding portion 220a is accommodated in the curvedly recessed portion 220b and presses the curvedly recessed portion 220b, such that the above-mentioned supporting force may be generated.

For example, as illustrated in FIGS. 1 to 3, the curvedly protruding portion 220a may be provided at the lower end of the protruding region 220 of the first link part 200a, and the curvedly recessed portion 220b may be provided at the upper end (e.g., an upper surface) of the protruding region 220 of the second link part 200b.

Hereinafter, the detailed shape of the protruding region 220 of the link part 200 will be described. However, in the device for assisting muscular strength according to an embodiment of the present disclosure, the detailed shape of the protruding region 220 is not necessarily limited to a shape to be described below.

With reference to FIGS. 1 to 4, the protruding region 220 may include a first extension section 222 extending in a direction intersecting the upward/downward direction. In this example case, the curvedly recessed portion 220b may be provided on an upper surface of the first extension section 222 of the protruding region 220 of the second link part 200b.

The protruding region 220 may include a connection section 224 extending downward from one side of the first extension section 222, and a second extension section 226 connected to a lower end of the connection section 224 and extending in the direction intersecting the upward/downward direction. In this example case, the curvedly protruding portion 220a may be provided on a lower surface of the second extension section 226 of the protruding region 220 of the first link part 200a.

For example, as illustrated in FIGS. 1 to 4, the first extension section 222 and the second extension section 226 may each have a shape extending in an approximately leftward/rightward direction, and a longitudinal direction of the first extension section 222 and a longitudinal direction of the second extension section 226 may be parallel to each other.

According to the example embodiment of the present disclosure, the connection section 224 may include sections inclinedly extending downward. More specifically, the connection section 224 may include a first connection section 224a inclinedly extending downward from one longitudinal end of the first extension section 222, and a second connection section 224b inclinedly extending downward from the other longitudinal end of the first extension section 222. A lower end of the first connection section 224a and a lower end of the second connection section 224b may meet together. For example, a combination of the first extension section 222, the first connection section 224a, and the second connection section 224b may have an approximately triangular shape symmetric in the leftward/rightward direction. In this example case, the second extension section 226 may be connected to the lower end of the first connection section 224a and the lower end of the second connection section 224b.

With continued reference to FIGS. 1 to 4, protruding connection portions 226a may be provided at one longitudinal side of the second extension section 226 and have a shape protruding in the upward/downward direction, and the second wire parts 320 may be respectively connected to the protruding connection portions 226a. In this example case, according to an embodiment of the present disclosure, one side of the second-first wire 321 and one side of the second-second wire 322 may be respectively connected to the protruding connection portions 226a. According to an embodiment of the present disclosure, in addition to the protruding connection portions 226a, the device 10 for assisting muscular strength may further include portions connected to one side of the first wire part 310 or one side of the second wire part 320, formed on the base region 210 of the first link part 200a or the second link part 200b, and protruding in the upward/downward direction (see FIGS. 1 to 4). However, for convenience of description, in the present specification, the above-mentioned portions will be considered as partial portions of the base region 210 and described.

As described above, the second wire parts 320 may be respectively provided on the left and right regions of the first wire part 310. Therefore, the protruding connection portions 226a, to which one side of the second wire part 320 is connected, may also be respectively provided on the left and right regions of the first wire part 310. For example, the protruding connection portions 226a may be respectively provided at two opposite longitudinal sides of the second extension section 226. The lower end of the connection section 224 (i.e., a portion where the first connection section and the second connection section meet together) may be provided between the two protruding connection portions 226a respectively provided at the two opposite longitudinal sides of the second extension section 226.

The plurality of first-first wires 311 provided in the device 10 for assisting muscular strength according to an embodiment of the present disclosure may be provided as separate components without being connected to one another. The plurality of first-second wires 312 may also be provided as separate components without being connected to one another. That is, according to the present disclosure, one first-first wire 311 may be configured to connect only the two adjacent link parts 200 among the plurality of link parts 200 provided in the device 10 for assisting muscular strength, and one first-second wire 312 may be configured to connect only the two adjacent link parts 200 among the plurality of link parts 200 provided in the device 10 for assisting muscular strength. These features may be identified from the state in FIGS. 1 to 3 in which the plurality of first-first wires 311 are spaced apart from one another in the upward/downward direction based on the base region 210, and the plurality of first-second wires 312 are spaced apart from one another in the upward/downward direction based on the base region 210.

It is noted that the structure of the device 10 for assisting muscular strength described in the present specification has been described with reference to the example case in which the wearer wears the device 10 for assisting muscular strength and may stand without bending the waist forward or rearward, unless otherwise expressly stated. As can be apparent to one of ordinary skill in the pertinent art, the example embodiment described and illustrated in FIGS. 1 to 4 may be flipped in the upward/downward direction to provide an equivalent embodiment with equivalent capabilities.

The present disclosure has been described with reference to the limited example embodiments and the drawings, but the present disclosure is not necessarily limited thereby. The present disclosure may be carried out in various forms by those skilled in the art, to which the present disclosure pertains, within the technical spirit of the present disclosure and scopes equivalent to the appended claims.

What is claimed is:

1. A device for assisting muscular strength, the device comprising:
a support part including a section extending along a straight or curved line;
link parts including a first link part having one side fixed to the support part, and a second link part provided below the first link part; and
wire parts configured to connect the first link part and the second link part, wherein the first and second link parts each comprises:
a base region, and
a protruding region protruding downward from the base region, and wherein the wire parts comprise:
a first wire part having a first side connected to the base region of the first link part and a second side connected to the base region of the second link part, and
a second wire part having a first side connected to the base region of the second link part and a second side connected to the protruding region of the first link part.

2. The device of claim 1, wherein when a direction in which the first link part and the second link part face the support part is defined as a forward direction, the first wire part comprises:
a first-first wire configured to connect a front region of the base region of the first link part and a front region of the base region of the second link part; and
a first-second wire provided to face the first-first wire with the protruding region of the first link part interposed therebetween.

3. The device of claim 1, wherein when a direction in which the first link part and the second link part face the support part is defined as a forward direction, the second wire part comprises:
a second-first wire configured to connect the protruding region of the first link part and a portion of the base region of the second link part that is positioned forward of the protruding region of the first link part; and
a second-second wire configured to connect the protruding region of the first link part and a portion of the base region of the second link part that is positioned rearward of the protruding region of the first link part.

4. The device of claim 1, wherein the second wire part is duplicated and respectively provided on left and right regions of the first wire part.

5. The device of claim 4, wherein the second wire part provided on the right region of the first wire part and the second wire part provided on the left region of the first wire part are structured to be symmetric with respect to the first wire part.

6. The device of claim 3, wherein through-holes are respectively, in an upward/downward direction, through the base region of the first link part and the base region of the second link part,
wherein any one of a lower end of the protruding region of the first link part and an upper end of the protruding region of the second link part includes a curvedly protruding portion protruding while having a curved shape, and
wherein another of the lower end of the protruding region of the first link part and the upper end of the protruding region of the second link part includes a curvedly recessed portion having a shape corresponding to the curvedly protruding portion.

7. The device of claim 6, wherein the curvedly protruding portion is a ball joint member.

8. The device of claim 6, wherein the curvedly protruding portion is provided at the lower end of the protruding region of the first link part, and the curvedly recessed portion is provided at the upper end of the protruding region of the second link part.

9. The device of claim 8, wherein the protruding region includes a first extension section extending in a direction intersecting the upward/downward direction, and the curvedly recessed portion is provided on an upper surface of the first extension section of the protruding region of the second link part.

10. The device of claim 9, wherein the protruding region further comprises:
a connection section extending downward from one side of the first extension section; and
a second extension section connected to a lower end of the connection section and extending in the direction intersecting the upward/downward direction, and
wherein the curvedly protruding portion is provided on a lower surface of the second extension section of the protruding region of the first link part.

11. The device of claim 10, wherein a longitudinal direction of the first extension section and a longitudinal direction of the second extension section are parallel to each other.

12. The device of claim 10, wherein the connection section comprises:
a first connection section inclinedly extending downward from one longitudinal end of the first extension section; and
a second connection section inclinedly extending downward from another longitudinal end of the first extension section, and
wherein the second extension section is connected to a lower end of the first connection section and a lower end of the second connection section.

13. The device of claim 11, wherein protruding connection portions, to which the second wire part is connected, are provided at one longitudinal side of the second extension section, and
wherein one side of the second-first wire and one side of the second-second wire are respectively connected to the protruding connection portions.

14. The device of claim 13, wherein the protruding connection portions are respectively provided at two opposite longitudinal sides of the second extension section, and the lower end of the connection section is provided between the two protruding connection portions respectively provided at the two opposite longitudinal sides of the second extension section.

15. The device of claim 1, wherein the support part is configured to be tightly attached to or in contact with a wearer's waist.

16. A support device, the device comprising:
a support part;
a first link part;
a second link part adjacent the first link part, wherein the first and second link parts each comprises:
a base region, and
a protruding region protruding from the base region,
wherein the first link part has a first first-link-part side of the base region of the first link part that is attached to the support part, and wherein the second link part has a first second-link-part side of the base region of the second link part that is attached to the support part at a position spaced apart from where the first first-link-part side of the first link part is attached to the support part; and
wire parts connecting the first link part to the second link part, wherein the wire parts comprise:
a first wire part having a first first-wire-part end connected to the base region of the first link part and a second first-wire-part end connected to the base region of the second link part, and
a second wire part having a first second-wire-part end connected to the base region of the second link part and a second second-wire-part end connected to the protruding region of the first link part.

17. The device of claim 16, wherein the protruding region extends perpendicularly from the base region, wherein the base region has a through-hole opening to a top portion of the protruding region, wherein the second wire part extends over the through-hole opening of the second link part, and wherein at least part of the protruding region of the first link part extends into the through-hole opening of the second link part.

18. The device of claim 17, wherein the protruding region protrudes downward from the base region, wherein base region and the protruding region are integral, wherein the protruding region has a generally triangular shape, and wherein the support part is configured to attach to a human body.

19. A human support device, the device comprising:
a support part configured to attach to a human body;
a first link part;
a second link part adjacent the first link part, wherein the first and second link parts each comprises:
a base region, and
a protruding region protruding from the base region, wherein the first link part has a first first-link-part side of the base region of the first link part that is attached to the support part, and wherein the second link part has a first second-link-part side of the base region of the second link part that is attached to the support part at a position spaced apart from where the first first-link-part side of the first link part is attached to the support part; and
wire parts connecting the first link part to the second link part, wherein the wire parts comprise:
a front-first wire part extending generally vertically adjacent to the support part between the protruding region of the first link part and the support part, the front-first wire part having a first front-first-wire-part end connected to the base region of the first link part and a second front-first-wire-part end connected to the base region of the second link part,
a rear-first wire part extending generally vertically adjacent to the protruding region of the first link part, the rear-first wire part having a first rear-first-wire-part end connected to the base region of the first link part and a second rear-first-wire-part end connected to the base region of the second link part,
a left-front-second wire part extending generally horizontally adjacent a left side of the protruding region of the first link part between the base region of the first link part and the base region of the second link part, the left-front-second wire part having a first left-front-second-wire-part end connected to a left-front side of the base region of the second link part and a second left-front-second-wire-part end connected to the protruding region of the first link part,
a left-rear-second wire part extending generally horizontally adjacent the left side of the protruding region of the first link part between the base region of the first link part and the base region of the second link part, the left-rear-second wire part having a first left-rear-second-wire-part end connected to a left-rear side of the base region of the second link part and a second left-rear-second-wire-part end connected to the protruding region of the first link part,
a right-front-second wire part extending generally horizontally adjacent a right side of the protruding region of the first link part between the base region of the first link part and the base region of the second link part, the right-front-second wire part having a first right-front-second-wire-part end connected to a right-front side of the base region of the second link part and a second right-front-second-wire-part end connected to the protruding region of the first link part, and
a right-rear-second wire part extending generally horizontally adjacent the right side of the protruding region of the first link part between the base region of the first link part and the base region of the second link part, the right-rear-second wire part having a first right-rear-second-wire-part end connected to a right-rear side of the base region of the second link part and a second right-rear-second-wire-part end connected to the protruding region of the first link part.

20. The device of claim 19, wherein the protruding region extends perpendicularly from the base region, wherein the base region has a through-hole opening to a top portion of the protruding region, wherein at least part of the protruding region of the first link part extends into the through-hole opening of the second link part, wherein the protruding region protrudes downward from the base region, wherein base region and the protruding region are integral, and wherein the protruding region has a generally triangular shape.
